(19) United States
(12) Patent Application Publication
(10) Pub. No.: US 2010/0272839 A1
(43) Pub. Date: Oct. 28, 2010

(54) TREATMENT OF FATTY SCALP WITH A LYSATE OF BIFIDOBACTERIUM SPECIES

(75) Inventors: Audrey GUENICHE, Rueil-Malmaison (FR); Dominique Bernard, Vanves (FR); Isabelle Castiel, Nice (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/607,142

(22) Filed: Oct. 28, 2009

Related U.S. Application Data
(60) Provisional application No. 61/111,426, filed on Nov. 5, 2008.

(30) Foreign Application Priority Data
Oct. 28, 2008 (FR) 08 57333

(51) Int. Cl. A61K 35/74; A61P 17/00

(52) U.S. Cl. 424/780

(57) ABSTRACT

The present invention treats a fatty dandruff state of the scalp with a lysate of at least one microorganism of the genus Bifidobacterium species and/or a fraction thereof.

### CROSS-REFERENCE TO RELATED APPLICATIONS

This application claims priority to U.S. provisional application Ser. No. 61/111,426, filed Nov. 5, 2008; and to French patent application 08 57333, filed Oct. 28, 2008, both incorporated herein by reference.

### FIELD OF THE INVENTION

The present invention mainly aims to propose a novel active for the prevention and/or treatment of disorders, notably aesthetic, associated with fatty scalp.

The scalp is epidermis that is constantly being renewed, like the rest of the cutaneous tissue, and has numerous sebaceous glands.

### BACKGROUND OF THE INVENTION

The secretion of sebum is a phenomenon that is normal and is useful to the skin and to the hair. Sebum protects the scalp and provides shine of the hair by lubricating the cuticle.

Unfortunately, hypersecretion of sebum, or seborrhoea, can prove unpleasant, and cause sensations of discomfort, aesthetic disorders, or even a skin pathology. Thus, excessive secretion of sebum can lead to greasy skin, or even acne, and promote the development of a fatty dandruff state of the scalp or greasy dandruff.

The fatty dandruff states are states that are chronic, common, recurring, and socially incapacitating owing to their manifest unsightliness. A great many factors may amplify these phenomena and then lead to the development of additional problems such as inflammatory states of the scalp. These fatty dandruff states and/or inflammatory states of the scalp are reflected in a change in the barrier function of the epidermis. Furthermore, these states can cause sensations of itching or pruritus, leading to scratching behaviour that exacerbates the phenomenon of development of dandruff.

For example, stress, the winter season or colonization of the skin or of the hair follicles by the yeast Malassezia sp. are factors that intensify these states in most subjects.

The yeast Malassezia sp. represents about 45% of the normal commensal flora on the surface of the scalp in subjects without dandruff, but can represent 75% of the flora in the case of dandruff, and up to 85% in the case of associated seborrhoeic dermatitis. Other microorganisms present on the surface of the scalp are micrococci and Propionibacterium.

Now, it was recently demonstrated that yeasts of the Malassezia type possess considerable lipase activity, leading to hydrolysis of the triglycerides of the sebum to fatty acids. These fatty acids are then capable of causing dandruff states in persons who are sensitive, i.e. whose barrier function has been impaired, and who are therefore more particularly susceptible to the destructive action of the fatty acids on the skin barrier.

Thus, greasy dandruff develops more easily when more sebum is present. Moreover, it is more likely to be pruriginous.

This aesthetic disorder can partly be neutralized by various local or systemic antifungal treatments. Thus, various preparations combining antifungals and anti-seborrhoeics have been proposed for treating severe fatty dandruff states. Treatments based on antifungals demonstrate some degree of efficacy on fatty dandruff states.

However, the efficacy of these treatments is only suspensive and requires rigorous follow-up on the part of the user (frequent use and sufficient time of application).

Consequently, numerous failures occur in the application of these treatments and can usually be ascribed to the following factors: poor adherence to the protocol; non-observance of the frequency of use; non-cosmetic appearance of the product, irritation of the washing basis; poor observance of the duration of application; tiredness.

### SUMMARY OF THE INVENTION

Therefore there is still a need for novel actives that can exert a beneficial cosmetic or therapeutic action on fatty scalp conditions.

There is also still a need for actives that make it possible to restore the ecoflora of the scalp and notably prevent excessive colonization of the scalp by Malassezia sp.

There is also a need for effective novel compositions for preventing and/or treating fatty scalp conditions, which are pleasant and comfortable to use, thus promoting compliance with the treatment.

There is also a need for novel actives capable of preventing and/or treating inflammatory states of fatty scalp, notably such as pruriginous states and seborrhoeic dermatitis.

The aim of the present invention is to satisfy these needs.

Thus, according to a first object, the invention relates to the cosmetic use of a lysate of at least one microorganism of the genus Bifidobacterium species or a fraction thereof for treating and/or preventing fatty scalp conditions.

According to a preferred embodiment, the invention relates to a cosmetic use of a lysate of at least one microorganism of the genus Bifidobacterium longum.

According to another object, the invention relates to a cosmetic method for treating and/or preventing fatty scalp conditions, comprising at least a step of administering to an individual in need thereof of a lysate of at least one microorganism of the genus Bifidobacterium species or a fraction thereof.

### DETAILED DESCRIPTION OF THE INVENTION

Unexpectedly, the inventors observed that certain probiotic microorganisms, in particular of the Bifidobacteria type, can prove particularly effective for improving and restoring fatty scalp conditions.

Thus, as follows from the data presented in the examples, the inventors notably characterized the capacity of certain of these microorganisms for stimulating the synthesis of a surprising number of proteins that may promote and reinforce the antimicrobial defenses of the epidermis.

In particular, the inventors demonstrated that a lysate of Bifidobacterium longum was able to stimulate the synthesis of proteins such as Ribonuclease 7 (Q9H1E1), dermcidin (P81605), prolactin-inducible protein (P12273), proteins S100 A8 and A9 (P05109 and P06702), and the protein histone (Q5R2W0), which are able to reinforce the defenses of the epidermis against excessive colonization by pathogenic microorganisms.

Thus, application of the cosmetic or dermatological or pharmaceutical compositions of the invention promotes a decrease in colonization of the scalp and of the hair follicles by Malassezia sp.

Consequently, the compositions of the invention can advantageously permit reestablishment of a balanced ecoflora through induction of proteins of epidermal defenses.

According to another advantage, use according to the invention can reduce and/or treat pruritus of the scalp due to the presence of irritant metabolites resulting from metabolism of the lipids of the sebum by Malassezia sp.

According to another of its aspects, the present invention relates to the use of a lysate of at least one microorganism of the genus Bifidobacterium species or a fraction thereof for preparing a composition, notably pharmaceutical or dermatological, intended for preventing and/or treating the inflammation of fatty scalp.

In particular, such a composition proves effective for treating pruritus of the scalp or seborrhoeic dermatitis of the scalp.

A composition of the invention can advantageously be used for preventing and/or treating skin infections and notably of the scalp by Malassezia sp.

In the sense of the present invention, the term “prevent” signifies reducing the risk of manifestation of the phenomenon in question.

According to another of its aspects, the invention relates to a notably cosmetic method for treating and/or preventing the aesthetic disorders of fatty scalp in an individual in need thereof, comprising at least one stage of administering to said individual of a lysate of at least one microorganism of the genus Bifidobacterium species or a fraction thereof.

A use according to the invention can, moreover, comprise the application of a lysate of at least one microorganism of the genus Bifidobacterium species or a fraction thereof, in combination with an effective amount of at least one additional microorganism, notably probiotic, and/or a fraction thereof, different from said lysate.

In the sense of the invention, the expression “different from said lysate” signifies that it is possible to distinguish, within the composition, either two different microorganisms or two different forms of one and the same microorganism. Thus, when the additional microorganism is of the genus Bifidobacterium species and corresponds to the same species as that representing the lysate required according to the invention, this additional microorganism is then present in a form other than a lysate.

A use according to the present invention can, moreover, comprise the application of a lysate of at least one microorganism of the genus Bifidobacterium species or a fraction thereof, and in combination with an effective amount of at least one active intended to reduce and/or correct excessive secretion of sebum, for example an antiseborrhoeic active notably as described below.

According to one embodiment, a use according to the invention can, moreover, comprise the application of a lysate of at least one microorganism of the genus Bifidobacterium species or a fraction thereof, in combination with an effective amount of at least one active intended to reduce and/or prevent and/or suppress dandruff, for example an antidandruff active notably as described below.

According to another of its aspects, the present invention relates to a cosmetic and/or dermatological composition that can be used for preventing and/or treating fatty scalp conditions, notably the fatty dandruff states of the scalp, comprising in a physiologically acceptable medium, at least one lysate of at least one microorganism of the genus Bifidobacterium species or a fraction thereof, in combination with an effective amount of at least one antiseborrhoeic active and/or at least one antidandruff active, notably as described below.

According to a variant embodiment of the invention, a lysate according to the invention can be used by the oral route.

According to another variant embodiment of the invention, the lysate according to the invention can be used topically.

As stated below, the compositions containing it are formulated to be compatible with the method of administration adopted.

The invention relates to the application of a lysate according to the invention in the form of a cosmetic or dermatological or pharmaceutical composition.

Fatty Dandruff State

As stated previously, a fatty scalp resulting from excessive secretion of sebum may be manifested by the development of greasy dandruff, or even pruritus reflecting inflammation of the epidermis.

Fatty dandruff states represent one of the forms of seborrhoeic dermatitis. Subjects affected by these conditions have an erythematous scalp covered with extensive, yellow, greasy scales which accumulate to form masses. They have a pruriginous scalp, and they often have burning sensations on the areas affected. These phenomena can be amplified by the presence of pathogenic microorganisms, notably Malassezia sp. These microorganisms, which have the characteristic that they release fatty acids from the sebum, can impair the barrier function of the epidermis and generate inflammatory states.

The seborrhoeic dermatitides can also affect regions of the face (median zones, areas around the eyes, ears), as well as the armpits.

In fatty dandruff states of the scalp, the skin barrier is unbalanced, its integrity is impaired and its ecoflora is disturbed. The skin of the scalp is irritated and pruriginous, brittle and less hydrated.

The application of a lysate of Bifidobacterium sp. according to the invention leads to reestablishment of the ecoflora and a decrease in pruritus. This decrease is reflected in reduction of the phases of scratching of the scalp and of the resultant impairment of the barrier function.

The skin is then less irritated and less pruriginous and the presence of greasy dandruff is reduced, or even eliminated.

The uses, methods and compositions according to the invention thus proved quite particularly effective:
for preventing and/or treating disorders, notably aesthetic, of the scalp linked to excessive excretion and/or secretion of sebum,
for preventing and/or treating fatty scalp, and notably fatty dandruff states of the scalp,
for preventing and/or treating greasy skin, for preventing and/or treating pruritus and/or seborrhoeic dermatitis of fatty scalp,
for reestablishing a balanced ecoflora of the fatty scalp, and
for improving and/or reestablishing the antimicrobial defenses of fatty scalp,
for improving the comfort of greasy skin and scalp, for preventing and/or treating inflammatory fatty scalp conditions.

Microorganisms

As stated previously, the microorganisms of the genus Bifidobacterium species used as actives according to the invention are employed in the form of a lysate.

A lysate commonly denotes a material obtained after destruction or dissolution of biological cells by a phenomenon called cell lysis, thus causing the release of the intracellular biological constituents naturally contained in the cells of the microorganism in question.

In the sense of the present invention, the term lysate is used without distinction to denote all of the lysate obtained by lysis of the microorganism in question or only a fraction thereof.

Thus, the invention relates to the application of a lysate of Bifidobacterium species and/or a fraction thereof.

The lysate used is therefore formed wholly or partly from the intracellular biological constituents and from the constituents of the cell walls and membranes.

More precisely, it contains the cellular cytoplasmic fraction containing the enzymes such as lactic acid dehydrogenase, phosphatases, phosphoketolases, and transaldolases and the metabolites. For purposes of illustration, the constituents of the cell walls are notably peptidoglycan, murein or mucopeptide and teichoic acid and the constituents of the cell membranes are glycerophospholipid compounds.

This cell lysis can be performed by various technologies, for example osmotic shock, thermal shock, with ultrasound, or under mechanical stress such as centrifugation.

According to a preferred embodiment, the lysate is obtained by disintegration by ultrasound.

More particularly, said lysate can be obtained according to the technology described in U.S. Pat. No. 4,464,362, and notably according to the following protocol.

A microorganism of the Bifidobacterium species type considered is cultivated anaerobically in a suitable culture medium, for example according to the conditions described in documents U.S. Pat. No. 4,464,362 and EP 0 043 128. When the stationary phase of development is reached, the culture medium can be inactivated by pasteurization, for example at a temperature of 60 to 65° C. for 30 min. The microorganisms are then collected by a conventional separation technique, for example membrane filtration, centrifuged and resuspended in a sterile NaCl solution at a physiological concentration. The lysate can be obtained by ultrasonic disintegration of said medium in order to release its cytoplasmic fractions, the fragments of cell wall and the products resulting from metabolism. Then all the components in their natural distribution are stabilized in a weakly acid aqueous solution.

In this way a lysate is generally obtained that has a concentration of the order of 0.1 to 50%, in particular from 1 to 20%, and notably about 5% by weight of active substance(s) relative to its total weight.

The lysate can be used in various forms, in the form of a solution or in a pulverulent form.

A microorganism belonging to the genus Bifidobacterium species is more particularly selected from the species: Bifidobacterium longum, Bifidobacterium bifidum, Bifidobacterium breve, Bifidobacterium animalis, Bifidobacterium lactis, Bifidobacterium infantis, Bifidobacterium adolescentis or Bifidobacterium pseudocatenulatum, and mixtures thereof.

The species Bifidobacterium longum is quite particularly suitable for the invention.

It can advantageously be the lysate registered under the INCI name: Bifidat ferment Lysate, under the EINECS name: Bifidobacterium longum, under the EINECS No.: No. 306-168-4 and under the CAS No.: No. 96507-89-0.

The product marketed under the designation Repair Complex CLR® by the company K. RICHTER GmbH and that is formed of an inactivated lysate of the species Bifidobacterium longum falls within the scope of the invention.

The active forming the lysate and belonging to the genus Bifidobacterium species can be formulated in a composition at a rate of at least 0.0001% (expressed in dry weight), in particular at a rate from 0.001 to 20% and more particularly at a rate from 0.01 to 2% of dry weight of active substance relative to the total weight of the carrier or of the composition containing it.

In the particular case when the microorganism(s) is (are) formulated in compositions for oral administration, the concentration of microorganism(s) can be adjusted so as to correspond to doses (expressed in equivalent of microorganism) ranging from 5.10² to 10¹³ CFU/d and in particular from 10⁵ to 10″ CFU/d.

According to a variant of the invention, a lysate suitable for the invention is used in combination with at least one other microorganism.

Thus, the invention relates to the use, in addition to a microorganism of the genus Bifidobacterium species, of at least an effective amount of at least one additional microorganism, notably of probiotic type, and/or a fraction thereof and/or one of its metabolites, different from said lysate.

In the sense of the present invention, “probiotic microorganism” means a live microorganism which, when it is consumed in an appropriate amount, has a positive effect on the health of its host “Joint FAO/WHO Expert Consultation on Evaluation of Health and Nutritional Properties of Probiotic in Food Including Powder Milk with Live Lactic Acid Bacteria, 6 Oct. 2001”, and which can in particular improve the intestinal microbial balance.

An additional microorganism suitable for the invention can be selected notably from the ascomycetes such as Saccharomyces, Yarrowia, Kluyveromyces, Torulaspora, Schizosaccharomyces pombe, Debaryomyces, Candida, Pichia, Aspergillus and Penicillium, bacteria of the genus Bifidobacterium, bacteroides, Fusobacterium, Melissococcus, Propionibacterium, Enterococcus, Lactococcus, Staphylococcus, Peptostreptococcus, Bacillus, Pediococcus, Micrococcus, Leuconostoc, Weissella, Aerococcus, Oenococcus, Lactobacillus, and mixtures thereof.

As ascomycetes quite particularly suitable for the present invention, we may mention in particular Yarrowia lipolytica and Kluyveromyces lactis, as well as Saccharomyces cerevisiae, Torulaspora, Schizosaccharomyces pombe, Candida and Pichia.

Specific examples of additional probiotic microorganisms are Lactobacillus acidophilus, Lactobacillus alimentarius, Lactobacillus curvatus, Lactobacillus delbrueckii subsp. lactis, Lactobacillus gasseri, Lactobacillus johnsonii, Lactobacillus reuteri, Lactobacillus rhamnosus (Lactobacillus GG), Lactobacillus sake, Lactobacillus paracasei, Lactococcus lactis, Streptococcus thermophilus, Staphylococcus carnosus, Staphylococcus xylosus, and mixtures thereof.

More particularly, they are probiotic microorganisms from the group of the lactic acid bacteria, such as notably Lactobacillus. As an example of these lactic acid bacteria, we may more particularly mention Lactobacillus johnsonii, Lactobacillus reuteri, Lactobacillus rhamnosus, and mixtures thereof.

As stated previously, the additional microorganism may or may not be of the same species as that forming the lysate. However, when it is of the same species, it is then present in a form other than a lysate, for example in a live form.

The species that are quite particularly suitable are Lactobacillus johnsonii, notably the strain deposited in accordance with the Budapest treaty with the Institut Pasteur (28 rue du Docteur Roux, F-75024 Paris cedex 15) under the following designation CNCM 1-1225. It goes without saying that a lysate of the invention is used in an effective amount.

“Effective amount” means, in the sense of the present invention, an amount that is sufficient to obtain the expected effect.

In general, a composition for topical application according to the invention generally comprises from 0.0001 to 30%, in particular from 0.001 to 15% and more particularly from 0.1 to 10% of one or more additional notably probiotic, microorganisms.

This or these microorganism(s) can be included in a composition according to the invention in a live form, semi-active form or inactivated, dead form.

It or they can also be included in the form of fractions of cellular components or in the form of metabolites. The microorganism(s), metabolite(s) or fraction(s) can also be included in the form of a powder, a liquid, a culture supernatant or a fraction thereof, diluted or not, or concentrated or not.

In the case when the microorganisms are formulated in a composition in live form, the quantity of live microorganisms can vary from 10³ to 10¹⁵ CFU/g, in particular from 10⁵ to 10¹⁵ CFU/g and more particularly from 10⁷ to 10¹² CFU/g of microorganisms per gram of composition.

The compositions according to the invention can be in all the galenical forms normally available for the method of administration adopted.

The carrier can be of varying nature depending on the type of composition considered.

More particularly with regard to the compositions intended for topical administration, these can be an aqueous, aqueous-alcoholic or oily solution, a dispersion of the solution type or dispersion of the lotion or serum type, an emulsion of liquid or semi-liquid consistency of the milk type, obtained by dispersion of an oil phase in an aqueous phase (O/W) or vice versa (W/O), or a suspension or emulsion of soft, semi-solid or solid consistency, of the cream type, aqueous or anhydrous gel, or microemulsions, microcapsules, microparticles, or vesicular dispersions of the ionic and/or non-ionic type.

These compositions are prepared according to the usual methods.

These compositions can notably constitute creams for cleaning, protection, treatment or care, lotions, gels or mousses for care of the scalp, such as lotions for cleaning or for disinfection or compositions for the bath.

The compositions according to the invention can also consist of solid preparations as constituents of soaps or cleaning bars.

They can also be used for the scalp in the form of solutions, creams, gels, emulsions, mousses or in the form of aerosol compositions also containing a propellant under pressure.

When the composition of the invention is an emulsion, the proportion of the fat phase can be from 5 to 80%, and preferably from 10 to 50% by weight relative to the total weight of the composition. The oils, the emulsifiers and the co-emulsifiers used in the composition in the form of emulsion are selected from those classically used in the area of cosmetics and/or dermatology. The emulsifier and the co-emulsifier can be contained in the composition in a proportion ranging from 0.3 to 30%, and preferably from 0.5 to 20% by weight relative to the total weight of the composition.

When the composition of the invention is a solution or an oily gel, the fatty phase can represent more than 90% of the total weight of the composition.

As is known, the galenical forms intended for topical administration can also contain additives that are usual in the cosmetic, pharmaceutical and/or dermatological field, such as hydrophilic or lipophilic gelling agents, hydrophilic or lipophilic actives, preservatives, antioxidants, solvents, perfumes, fillers, filters, odour absorbers and colorants. The amounts of these various additives are those classically used in the field in question, for example from 0.01 to 20% of the total weight of the composition. Depending on their nature, these additives can be introduced in the fat phase and/or in the aqueous phase.

As fats for use in the invention, we may mention mineral oils, for example hydrogenated polyisobutene and vaseline oil, vegetable oils, for example a liquid fraction of shea butter, sunflower oil and apricot almond oil, animal oils, for example perhydrosqualene, synthetic oils notably purcelline oil, isopropyl myristate and ethyl hexyl palmitate, unsaturated fatty acids and fluorinated oils, for example perfluoropolyethers. It is also possible to use fatty alcohols, fatty acids for example stearic acid and for example waxes, notably paraffin wax, carnauba wax and beeswax. It is also possible to use silicone compounds such as silicone oils and for example cyclomethicone and dimethicone, waxes, resins and silicone rubbers.

As emulsifiers that can be used in the invention, we may mention for example glycerol stearate, polysorbate 60, the cetylstearyl alcohol/ethoxylated cetylstearyl alcohol mixture with 33 moles of ethylene oxide sold under the designation Sinnowax AO® by the company HENKEL, the mixture of PEG-6/PEG-32/Glycol Stearate sold under the designation Tefose® 63 by the company GATTEFOSSE, PPG-3 myristyl ether, silicone emulsifiers such as cetyldimethicone copolyol and sorbitan mono- or tristearate, the stearate of PEG-40, the ethoxylated sorbitan monostearate (20OE).

As solvents for use in the invention, we may mention the lower alcohols, notably ethanol and isopropanol, propylene glycol.

A composition of the invention can also contain, advantageously, a thermal and/or mineral water, notably selected from Vittel water, the waters of the Vichy basin and the water from Roche Posay.

As hydrophilic gelling agents, we may mention the carboxylic polymers such as carbomer, the acrylic copolymers such as the acrylate/alkylacrylate copolymers, the polyacrylamides and notably the mixture of polyacrylamide, C13-14-Isoparaffine and Laureth-7 sold under the name Sepigel 305® by the company SEPPIC, the polysaccharides such as the cellulosic derivatives such as the hydroxyalkylcelluloses and in particular hydroxypropylcellulose and hydroxyethylcellulose, the natural gums such as guar gum, carob gum and xanthan gum and the clays.

As lipophilic gelling agents, we may mention the modified clays such as the bentones, the metal salts of fatty acids such as aluminium stearates and hydrophobic silica, or ethylcellulose and polyethylene.

In the case when a combination according to the invention is used by the oral route, the use of an ingestible carrier is preferred.

The ingestible carrier can be of various kinds depending on the type of composition considered.

Notably the following are suitable as food or pharmaceutical carriers: milk, yoghurt, cheese, fermented milks, milk-based fermented products, ices, products based on fermented cereals, milk-based powders, formulas for children and babies, food products of the confectionery type, chocolate, cereals, feed for animals in particular domestic animals, tablets, capsules or lozenges, oral supplements in dry form and oral supplements in liquid form.

A microorganism of the invention, and/or a fraction thereof, can moreover be formulated with the usual excipients and components for such oral compositions or food supplements, namely notably fat and/or aqueous components, humectants, thickeners, preservatives, texture agents, flavour agents and/or enrobing agents, antioxidants, preservatives and colorants that are usual in the food products area.

The formulating agents and excipients for oral compositions, and notably for food supplements, are known in this field and will not be described in detail here. For ingestion, numerous embodiments of oral compositions and notably of food supplements are possible. They are formulated by the usual methods to produce coated pills, capsules, gels, controlled-release hydrogels, emulsions, tablets, capsules.

In particular, the microorganism according to the invention can be incorporated in any other forms of food supplements or of enriched foods, for example food bars, or powders, compacted or not. The powders can be diluted with water, in soda, milk products or soya derivatives, or can be incorporated in food bars.

According to a particular embodiment, the additional microorganisms considered according to the invention can be formulated within compositions in encapsulated form for significantly improving their survival time. In such a case, the presence of a capsule can in particular slow down or prevent the degradation of the microorganism in the gastrointestinal tract.

Regardless of the method of administration considered, a lysate of the invention can advantageously be combined with at least one other active.

Thus, a topical or oral composition, or a combination according to the invention can moreover contain at least one antiseborrhoeic active and/or at least one antidandruff active.

Such a formulation advantageously makes it possible to amplify the beneficial effects of a lysate of the invention.

“Antiseborrhoeic active” means a compound capable of regulating the activity of the sebaceous glands.

A antiseborrhoeic active suitable for the invention can notably be selected from retinoic acid, benzoyl peroxide, sulphur, vitamin B6 (or pyridoxine), selenium chloride, samphire; mixtures of extracts of cinnamon, of tea and of octanoylglycine such as Sepicontrol A5 TEA® from Seppic; the mixture of cinnamon, of sarcosine and of octanoylglycine, marketed notably by the company SEPPIC under the trade name Sepicontrol A5®; zinc salts such as zinc gluconate, zinc pyrrolidone carboxylate (or zinc pidolate), zinc lactate, zinc aspartate, zinc carboxylate, zinc salicylate, zinc cysteate; derivatives of copper and in particular copper pidolate such as Cuivridone® from Solabia; extracts of plants of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, all marketed for example by the company MARUZEN; extracts of meadowsweet (Spiraea ulamaria) such as that sold under the name Sébonormine® by the company Silab; extracts of alga Laminaria saccharina such as that sold under the name Phlorogine® by the company Biotechmarine; the mixtures of extracts of burnet roots (Sanguisorba officinalis/Poterium officinale), of ginger rhizomes (Zingiber officinalis) and of cinnamon bark (Cinnamomum cassia) such as that sold under the name Sebustop® by the company Solabia; linseed extracts as sold under the name Linumine® by the company Lucas Meyer; extracts of Phellodendron such as those sold under the name Phellodendron extract BG by the company Maruzen or Oubaku liquid B by the company Ichimaru Pharcos; the mixtures of argan oil, extract of Serenoa serrulata (saw palmetto) and sesame seed extract such as that sold under the name Regu SEB® by the company Pentapharm; the mixtures of extracts of epilobe, of Terminalia chebula, of nasturtium and of bioavailable zinc (microalgae) such as that sold under the name Seborilys® by the company Green Tech; the extracts of Pygeum afrianum such as that sold under the name Pygeum afrianum sterolic lipid extract by the company Euromed; the extracts of Serenoa serrulata such as those sold under the name Viapure Sabal by the company Actives International, or those sold by the company Euromed; the mixtures of extracts of plantain, of Berberis aquifolium and of sodium salicylate such as that sold under the name Seboclear® by the company Rahn; clove extract such as that sold under the name Clove extract Powder by the company Maruzen; argan oil such as that sold under the name Lipofructyl® by Laboratoires Sérobiologiques; filtrates of lactic protein such as that sold under the name Normaseb® by the company Sederma; extracts of alga Laminaria, such as that sold under the name Laminarghane® by the company Biotechmarine; oligosaccharides of alga Laminaria digitata such as that sold under the name Phycosaccharide AC by the company Codif; extracts of cane sugar such as that marketed under the name Policosanol® by the company Sabinsa; sulphonated shale oil, such as that sold under the name Ichtyol Pale® by the company Ichthyol; extracts of meadowsweet (Spiraea ulmaria) such as that sold under the name Cytobiol® Ulmaire by the company Libiol; sebacic acid, notably sold in the form of a gel of sodium polyacrylate under the name Sebosoft® by the company Sederma; glucomannans extracted from konjac tuber and modified with alkylsulphonate chains such as that sold under the name Biopol Beta by the company Arch Chemical; extracts of Sophora angustifolia, such as those sold under the name Sophora powder or Sophora extract by the company Bioland; extracts of Cinchona succirubra bark such as that sold under the name Red bark HS by the company Alban Muller; extracts of Quillaja saponaria such as that sold under the name Panama wood HS by the company Alban Muller; glycine grafted on undecylene chain, such as that sold under the name Lipacide UG OR by the company Seppic; the mixture of oleanolic acid and nordihydroguaiaretic acid, such as that sold in the form of a gel under the name AC.Net by the company Sederma; phthalimidoperoxyhexanoic acid; trialkyl(C₁₂-C₁₃) citrate sold under the name COSMACOL® ECI by the company Sasol; trialkyl(C₁₄-C₁₅) citrate sold under the name COSMACOL® ECL by the company Sasol; 10-hydroxydecanoic acid, and notably mixtures of 10-hydroxydecanoic acid, of sebacic acid and of 1,10-decanediol such as that sold under the name Acnacidol® BG by the company Vincience; and mixtures thereof.

The antiseborrhoeic active is for example present in a content ranging from 0.1 to 10%, preferably from 0.1 to 5%, and more preferably from 0.5 to 3% by weight, relative to the total weight of the composition.

“Antidandruff active” means a compound capable of preventing the development of dandruff, reducing its extent and/or causing it to disappear completely.

An antidandruff active suitable for the invention can notably be selected from:
the pyridinethione salts, notably the calcium, magnesium, barium, strontium, zinc, cadmium, tin and zirconium salts. The zinc salt of pyridinethione is particularly preferred. The zinc salt of pyridinethione is notably marketed under the designation Zinc omadine by the company OLIN.
the trihalogen carbamides of formula:

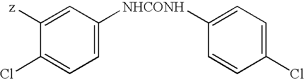

in which Z represents a halogen atom such as chlorine or a C₁-C₄ trihalogen-alkyl group such as CF₃.
triclosan represented by the formula:

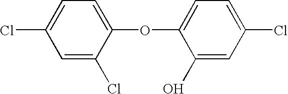

the azole compounds such as climbazole, ketoconazole, clotrinazole, econazole, isoconazole and miconazole.
the antifungal polymers such as amphotericin B or nystatin.
selenium sulphides, in particular those of formula SₓSe₈₋ₓ, x being in the range from 1 to 7.
sulphur in its various forms, cadmium sulphide, allantoin, coal or wood tars and their derivatives, in particular cade oil, salicylic acid, undecylenic acid, fumaric acid, allylamines such as terbinafine.

As preferred examples of antidandruff agents, we may notably mention pyrithione zinc, salicylic acid, selenium disulphide, and mixtures thereof.

A composition according to the invention advantageously comprises from 0.001 to 10% by weight of antidandruff agent(s), preferably from 0.1 to 5% by weight, even more preferably from 0.2 to 2% by weight, relative to the total weight of the composition.

The topical or oral compositions, or combinations according to the invention can also contain, as an additional active, an active notably intended to reinforce the skin barrier, such as an active promoting for example the maturation of the stratum corneum, for example a hydrating active, a prodesquamation agent, or an active promoting the delayed differentiation of the epidermis and the associated enzymatic activities.

As actives that are used conventionally, we may mention vitamins B3, B5, B6, B8, C, E, or PP, niacin, the carotenoids, the polyphenols and minerals such as zinc, calcium, and magnesium. “Prodesquamation agent” means any compound that is able to act:
either directly on desquamation, promoting exfoliation, such as the β-hydroxyacids, in particular salicylic acid and its derivatives (including n-octanoyl-5-salicylic acid); the α-hydroxyacids, such as glycolic, citric, lactic, tartaric, malic or mandelic acids; urea and certain of its derivatives; gentisic acid; the oligofucoses; cinnamic acid; dioic acid; extract of Saphora japonica; resveratrol; detergents and certain derivatives of jasmonic acid;
and/or on the activities of the enzymes involved in the degradation of the corneodesmosomes, such as the stratum corneum chymotryptic enzyme (SCCE) or even other proteases (trypsin-like, chymotrypsin-like, cathepsin D) as well as other categories of hydrolases (e.g. glycosidases, ceramidases). We may mention the chelating agents of the mineral salts: EDTA; N-acyl-N,N′,N′-ethylenediaminetriacetic acid; aminosulphonic compounds and in particular (N-2-hydroxyethylpiperazine-N-2-ethane) sulphonic acid (HEPES); derivatives of 2-oxothiazolidine-4-carboxylic acid (procysteine); derivatives of alpha amino acids of the glycine type (such as described in EP-0 852 949, as well as sodium methyl glycine diacetate marketed by BASF under the trade name TRILON M); honey; sugar derivatives such as O-octanoyl-6-D-maltose and N-acetyl glucosamine; urea or some of its derivatives, for example Hydrovance; derivatives of C-Glycosides.

In particular, an antioxidant complex can be used as active, comprising vitamins C and E, and at least one carotenoid, notably a carotenoid selected from β-carotene, lycopene, astaxanthin, zeaxanthin and lutein, flavonoids such as catechins, hesperidin, proanthocyanidins and anthocyanins, ubiquinones, coffee extracts containing polyphenols and/or diterpenes, chicory extracts, extracts of ginkgo biloba, proanthocyanidin-rich grape extracts, pepper extracts, soya extracts, other sources of flavonoids possessing antioxidant properties, fatty acids, prebiotics, taurine, resveratrol, amino acids of selenium, precursors of glutathione.

Among the flavonoids, the catechins and the OPCs (procyanidolic oligomers) are preferably selected.

There can be at least one prebiotic or a mixture of prebiotics. More particularly, these prebiotics can be selected from the oligosaccharides, produced starting from glucose, galactose, xylose, maltose, sucrose, lactose, starch, xylan, hemicellulose, inulin, gums of the acacia type for example, or a mixture thereof. More particularly, the oligosaccharide comprises at least one fructo-oligosaccharide. More particularly, said prebiotic can comprise a mixture of fructo-oligosaccharide and inulin.

In the topical galenical forms, it is possible to use, more particularly, as hydrophilic actives, proteins or protein hydrolysates, amino acids, polyols notably from C₂ to C₁₀ such as glycerol, sorbitol, butylene glycol and polyethylene glycol, urea, allantoin, sugars and sugar derivatives, water-soluble vitamins, starch, bacterial or plant extracts such as those from Aloe vera.

As for the lipophilic actives, it is possible to use retinol (vitamin A) and its derivatives, tocopherol (vitamin E) and its derivatives, ceramides, essential and non-saponifiable oils (tocotrienol, sesamin, gamma oryzanol, phytosterols, squalenes, waxes, terpenes).

As actives that can more particularly be combined with the lysate in an oral formulation, we may also consider all the ingredients commonly used and/or permitted.

By way of illustration, we may mention vitamins, minerals, essential lipids, trace elements, polyphenols, flavonoids, phytooestrogens, antioxidants such as lipoic acid and coenzyme Q10, carotenoids, prebiotics, proteins and amino acids, mono- and polysaccharides, amino-sugars, phytosterols and triterpene alcohols of vegetable origin.

They are, in particular, vitamins A, C, D, E, PP and the group B vitamins. Among the carotenoids, preference is given to beta-carotene, lycopene, lutein, zeaxanthin and astaxanthin. The minerals and trace elements used in particular are zinc, calcium, magnesium, copper, iron, iodine, manganese, selenium, and chromium(III). Among the polyphenols, the polyphenols from grape, tea, olive, cocoa, coffee, apple, bilberry, elder, strawberry, cranberry, and onion are also used in particular. Preferably among the phytooestrogens, the isoflavones are used, in free form or glycosylated, such as genistein, daidzein, glycitein or also lignans, in particular those from flax and from schizandra chinensis. The amino acids or the peptides and proteins containing them, such as taurine, threonine, cysteine, tryptophan, methionine. Lipids preferably belonging to the group of oils containing mono- and polyunsaturated fatty acids such as oleic, linoleic, alpha-linolenic, gamma-linolenic, stearidonic acids, long-chain omega-3 fish fatty acids such as EPA and DHA, conjugated fatty acids obtained from plants or animals such as CLA (conjugated linoleic acid).

A method of cosmetic treatment of the invention can notably be applied by administering the cosmetic and/or dermatological compositions or combinations as defined above, according to the usual technique for use of said compositions. For example: applications of creams, gels, serums, and lotions on the scalp, application of a lotion on wetted hair or shampoos in the case of topical application.

A cosmetic method according to the invention can thus be applied by topical or oral administration, daily for example, of the lysate under consideration according to the invention.

A method according to the invention can comprise a single administration. According to another embodiment, administration is repeated for example 2 to 3 times daily for one day or more and generally for an extended duration of at least 4 weeks, or even 4 to 15 weeks, with one or more periods of interruption if required.

In the description and in the examples given below, unless stated otherwise, the percentages are percentages by weight and the ranges of values stated in the form “between . . . and . . . ” include the lower and upper limits stated.

The ingredients are mixed, before being formed, in the order and in conditions that are easily determined by a person skilled in the art.

The content and the nature of the ingredients employed in the compositions of the invention are adjusted by a person skilled in the art in such a way that the properties required for the compositions of the invention are not substantially affected.

The examples given below are for purposes of illustration and do not limit the scope of the invention.

### EXAMPLES

### Example 1

| Lotion for the scalp | wt. % |
|---|---|
| Powder of lysate of Bifidobacterium longum* | 5.00** |
| Antioxidant | 0.05 |
| Isopropanol | 40.0 |
| Preservative | 0.30 |
| Water | q.s. 100 |

*It is the lysate registered under the name INCI Bifidat ferment Lysate, used as a formulation with 5% by weight of active
**amount based on total product

### Example 2

| Milk for care of the scalp | wt. % |
|---|---|
| Powder of lysate of Bifidobacterium longum* | 5.00** |
| Glycerol stearate | 1.00 |
| Oil of cetylstearyl alcohol/ethoxylated cetylstearyl alcohol | 3.00 |
| at 30 moles OE (Sinnowax AO ® sold by the company | |
| HENKEL) | |
| Cetyl alcohol | 1.00 |
| Dimethicone (DC 200 Fluid ® sold by the company | 1.00 |
| DOW CORNING) | |
| Vaseline oil | 6.00 |
| Isopropyl myristate (Estol IMP 1514 ® sold by UNICHEMA) | 3.00 |
| Antioxidant | 0.05 |
| Glycerine | 20.00 |
| Preservative | 0.30 |
| Water | q.s. 100 |

*It is the lysate registered under the name INCI Bifidat ferment Lysate, used as a formulation with 5% by weight of active
**amount based on total product

### Example 3

| Gel for care of the scalp | wt. % |
|---|---|
| Powder of lysate of Bifidobacterium longum* | 5.00** |
| Hydroxypropylcellulose (Klucel H ® sold by the company | 5.00 |
| HERCULES) | 1.00 |
| Vitamin E | 2.50 |
| Antioxidant | 0.05 |
| Isopropanol | 40.00 |
| Preservative | 0.30 |
| Water | q.s. 100 |

*It is the lysate registered under the name INCI Bifidat ferment Lysate, used as a formulation with 5% by weight of active
**amount based on total product

### Example 4

| Milk for care of the scalp | wt. % |
|---|---|
| Powder of lysate of Bifidobacterium longum* | 5.00** |
| Glycerol stearate | 1.00 |
| Cetylstearyl alcohol/ethoxylated cetylstearyl alcohol | 3.00 |
| at 3 moles OE (Sinnovax AO ® sold by the company HENKEL) | |
| Cetyl alcohol | 1.00 |
| Dimethicone (DC 200 Fluid ® sold by the company | 1.00 |
| Dow Corning) | |
| Vaseline oil | 6.00 |
| Isopropyl myristate (Estol IPM 1514 ® sold by the | 3.00 |
| company Unichema) | |
| Glycerine | 20.00 |
| Preservative | 0.30 |
| Water | |

*It is the lysate registered under the name INCI Bifidat ferment Lysate, used as a formulation with 5% by weight of active
**amount based on total product

### Example 5

| Cream for care of the scalp | wt. % |
|---|---|
| Arachidyl behenyl alcohol/arachidylglusoside | 3.0 |
| Isohexadecane | 7.0 |
| Powder of lysate of Bifidobacterium longum* | 5.00** |
| Glycerine | 2.0 |
| Extract of Vitreoscilla filiformis | 3.0 |
| BHT | 0.05 |
| POB methyl | 0.1 |
| POB propyl | 0.05 |
| Water | q.s. 100 |

*It is the lysate registered under the name INCI Bifidat ferment Lysate, used as a formulation with 5% by weight of active
**amount based on total product

### Example 6

| Gel for care of the hair | wt. % |
|---|---|
| Powder of lysate of Bifidobacterium longum* | 5.00** |
| Copper citrate | 2.00 |
| Extract of Vitreoscilla filiformis | 3.00 |
| Antioxidant | 0.05 |
| Vitamin C | 2.50 |
| Antioxidant | 0.05 |
| Isopropanol | 40.00 |
| Preservative | 0.30 |
| Water | q.s. 100.00 |

*It is the lysate registered under the name INCI Bifidat ferment Lysate, used as a formulation with 5% by weight of active
**amount based on total product

### Example 7

### Evaluation of the Effects of a Lysate of Bifidobacterium longum on an Epidermis

The product tested is a lysate of Bifidobacterium longum in suspension, disintegrated (by ultrasound) in a weakly acid aqueous medium marketed under the name Repair Complex CLR®.

The active was tested on its own in a randomized, double-blind study.

Sixty-six women with dry skin were divided into two groups, placebo (n=33 group A), Repair Complex CLR® (n=33 group B). The treatments were applied topically for 58 days, the active being formulated at 10% of the test formulation. The carrier formulation is an oil/demineralized water emulsion Arlacel/myrj® containing 5% Parleam, 15% cyclopentasiloxane, 3% glycerol and 2% vaseline.

In the placebo formulation, the absence of lysate is compensated with water.

The subjects were evaluated on D1, D29, D43, and D57.

The variation of various cutaneous markers was investigated by differential proteomics on samples of isolated stratum corneum.

A sample is taken from the skin at times D1, D29, D43 and D57 by varnish stripping so as to remove only a portion of the stratum corneum, i.e. at most 4 to 5 layers of stratum corneum.

A cloth of nylon filter 41 μm type NY41 Millipore is applied on a previously defined area of the left leg. Then a transparent varnish with the reference 614254/T.D.

comprising: nitrocellulose 6.86 g; isopropanol 2.94 g; hypoallergenic alkyl resin 7.35 g; acetyl tributyl citrate 7.7 g; ethyl acetate 75.15 g; is spread by brush (15 mm), and is then left to dry for 15 min. The nylon cloth is then recovered using spring forceps, by pulling away the varnish stripping smartly.

The varnish strippings are stored at −20° C., flat in plastic bags.

These skin samples (varnish stripping of stratum corneum) were then analysed by proteomics using a technique called “isobaric marking”, to evaluate the expression of different proteins.

This “isobaric marking” technique or iTRAQ is based on the marking of tryptic peptides with a series of reagents, which are called isobaric as they all have a molecular weight of 145 Da, and form a covalent bond with the primary amines of the amino-terminal end or of the side chain of the lysine residues.

The marked peptides are detected by mass spectrometry with the intrinsic mass of the peptide plus 145 Da from the reagent. At the stage of fragmentation of the peptide, the contribution of each of the reagents is assessed from the release of ions (fragments) having different specific masses.

Such a method is described in more detail by Zieske (J. Exp. Bot., 2006, 57:1501) or Wiese et al. (Proteomics, 2007, 7:340).

The results of the analysis by proteomics showed that the lysate of Bifidobacterium longum stimulates the expression of various antimicrobial defense proteins of the epidermis such as Ribonuclease 7 (Uniref accession No. Q9H1E1), dermcidin (P81605), prolactin-inducible protein (P12273), proteins S100 A8 and A9 (P05109 and P06702), and the protein histone (Q5R2W0).

A lysate of the invention is therefore able to reinforce the barrier function of the epidermis, and treat and/or prevent the deleterious effects of excessive colonization of the epidermis by Malassezia sp., notably with respect to fatty scalp.

1. A method for treating a fatty scalp condition, the method comprising administering to an individual in need thereof, a lysate of at least one microorganism of the genus Bifidobacterium species or a fraction thereof.

2. The method according to claim 1, wherein the fatty scalp condition is fatty dandruff of the scalp.

3. The method according to claim 1, wherein the fatty scalp condition is seborrhoeic dermatitis of fatty scalp.

4. The method according to claim 1, wherein the fatty scalp condition is pruritus of fatty scalp.

5. The method according to claim 1, wherein said lysate reestablishes a balanced ecoflora of fatty scalp.

6. The method according to claim 1, wherein said lysate improves and/or restores the antimicrobial defenses of fatty scalp.

7. The method according to claim 1, wherein the microorganism of the genus Bifidobacterium species is selected from the group consisting of Bifidobacterium longum, Bifidobacterium bifidum, Bifidobacterium breve, Bifidobacterium animalis, Bifidobacterium lactis, Bifidobacterium infantis, Bifidobacterium adolescentis or Bifidobacterium pseudocatenulatum, and a mixture thereof.

8. The method according to claim 1, wherein the microorganism of the genus Bifidobacterium species is Bifidobacterium longum.

9. The method according to claim 1, wherein said lysate comprises from 0.1 to 50% by weight of active substance(s).

10. The method according to claim 1, wherein said lysate is administered topically or orally.

11. The method according to claim 1, further comprising administering with said lysate, an effective amount of at least one additional microorganism, and/or a fraction thereof, different from said lysate.

12. The method according to claim 11, wherein said additional microorganism is at least one ascomycete microorganism selected from the group consisting of Saccharomyces, Yarrowia, Kluyveromyces, Torulaspora, Schizosaccharomyces pombe, Debaromyces, Candida, Pichia, Aspergillus and Penicillium, bacteria of the genus Bifidobacterium, Bacteroides, Fusobacterium, Melissococcus, Propionibacterium, Enterococcus, Lactococcus, Staphylococcus, Peptostreptococcus, Bacillus, Pediococcus, Micrococcus, Leuconostoc, Weissella, Aerococcus, Oenococcus, Lactobacillus, and a mixture thereof.

13. A method for treating inflammation of a fatty scalp, the method comprising administering to an individual in need thereof a composition comprising a lysate of at least one microorganism of the genus Bifidobacterium species or a fraction thereof.

14. The method according to claim 1 or 13, wherein said lysate is in a composition that is in the form of an aqueous solution, an aqueous-alcoholic solution, an oily solution, a solution dispersion, a lotion dispersion, a serum dispersion, an O/W emulsion, an W/O emulsion, a cream, an aqueous gel, an anhydrous gel, a microemulsion, a microcapsule, a microparticle, or a vesicular dispersion.

15. A composition suitable for application to human skin, the composition comprising in a physiologically acceptable medium, an effective amount of a lysate of at least one microorganism of the genus Bifidobacterium species or a fraction thereof to treat a fatty scalp condition, and an effective amount of at least one antidandruff active.

16. A method for treating an aesthetic disorder of a fatty scalp in an individual in need thereof, the method comprising administering to said individual a lysate of at least one microorganism of the genus Bifidobacterium species or a fraction thereof.
